(19) United States
(12) Patent Application Publication
(10) Pub. No.: US 2006/0280713 A1
(43) Pub. Date: Dec. 14, 2006

(54) Method for producing porous moulded bodies containing alginate

(75) Inventor: Ralf Malessa, Essen (DE)

Correspondence Address: RANKIN, HILL, PORTER & CLARK, LLP, 925 EUCLID AVENUE, SUITE 700, CLEVELAND, OH, 44115-1405, US

(73) Assignee: Dr. Suwelack Skin & Health Care AG, Billerbeck (DE)

(21) Appl. No.: 10/557,562

(22) PCT Filed: May 21, 2004
(86) PCT No.: PCT/EP04/50883

§ 371 Date: Nov. 21, 2005

(30) Foreign Application Priority Data
May 23, 2003 (DE) 10323794.1

(51) Int. Cl. A61K 8/73; A61K 9/70

(52) U.S. Cl. 424/70.13; 424/443

(57) ABSTRACT

The invention concerns processes for the production of alginate-containing porous or sponge-like shaped articles, as well as the shaped articles obtained thereafter and their use.

### BACKGROUND OF THE INVENTION

1. Field of Invention

The invention relates to a process for the manufacture of alginate-containing porous and/or sponge-like shaped articles, as well as to the shaped articles available thereafter and their use. It is known that alkali alginates such as sodium alginate are water-soluble, whereas earth-alkaline alginates such as calcium alginates are insoluble in water. Thus, thin water-insoluble layers can be produced, for example, by spraying a thin sodium alginate film with a CaCl₂ solution. However, if manufacture of thicker layers is intended difficulties arise from the fact that the homogeneous incorporation of free calcium ions into a sodium alginate solution is made difficult by the large increase of the solution's viscosity, so that disjointed calcium alginate agglomerates are the result instead of uniform products.

2. Description of Related Art

To overcome this problem, U.S. Pat. No. 5,718,916 suggests, for example, to add a water-soluble complexing agent such as sodium citrate to the aqueous solution of the alginate composition. If, for example, an easily soluble calcium salt such as calcium chloride is subsequently added, the immediate precipitation of calcium alginate is prevented by the presence of the complexing agent, which is supposed to prevent the formation of insoluble calcium alginate globules in the product. However, the examples from the US printed publication are based of the scale of a few millimetres. The gelation time of the alginate solution only spans 30 to 60 seconds. If one tries to transfer this process to larger scales, it becomes apparent that the intended retardation by adding the complexing agent to the sodium alginate solution is not sufficient, and that a relatively large-format product with a high degree of homogeneity cannot be obtained. Moreover, the application of surface-active agents is obligatory in the above-mentioned process in order to achieve a sufficient dispersion of the components. However, the use of such surface-active agents may lead to intolerances, e.g. when applied to the skin. The fact that a sufficient retardation of the precipitation by the prior addition of the complexing agent is not achieved by the process of U.S. Pat. No. 5,718,916 is also confirmed in GB 2357765 by the same inventor, in which the process of U.S. Pat. No. 5,728,916 is therefore described as being disadvantageous. GB 2357765 discloses a process for the manufacture of water-insoluble alginate sponges or foam products for the manufacture of adhesive plasters or surgical products, in which water-soluble alginate is also being cross-linked by adding polyvalent metal ions in the presence of a foam-producing agent. A complexing agent is not present. In a preferred variant, ammonium hydroxide is present in order to decrease the viscosity of the calcium alginate. In the examples, calcium, for example, is added, followed by an acid. This process also has the disadvantage that the formation of the alginates cross-linked by means of the calcium ions proceeds relatively quickly after the acid has been added, so that homogeneous thick layers cannot be obtained. Moreover, the process necessitates the presence of a foaming agent, of surface-active agents, of a borate buffer as well as the above-mentioned ammonium compounds. This makes the process difficult to control, and the products obtained contain a plurality of components whose physiological effects must be taken into consideration.

In DE 202 19 666 U, pads are described for dermatological use comprising a polymeric carrier material, in particular alginic acid. Concrete examples relating to the manufacture of these pads cannot be gathered from this utility model.

Furthermore, DE 43 28 329 discloses freeze-dried biomatrices for the moisturization of the skin and for the topical transdermal application of pharmaceutical cosmetically active substances containing natural polysaccharides and modified polysaccharides. This publication also mentions the stabilization of the biomatrix by the formation of calcium alginate skeletons by the addition of calcium ions. It cannot be seen from this publication how homogeneous thicker alginate layers can be produced.

The manufacture of small-scale alginate sponges for oral use by adding soluble calcium salt (calcium gluconate) to a sodium alginate solution is described in WO 01/17377. However, for the reasons already mentioned above (no homogeneous incorporation of the calcium ions), this process is also not suitable for the manufacture of large-format alginate sponges. The application of active substances suggested therein is also made difficult due to the inhomogeneities that arise.

A process for the manufacture of polysaccharide foams, in particular based on an alginate, is known from WO 94/00512. In one embodiment, this patent specification also discloses a variant in which an insoluble carbonate or bi-carbonate salt are dispersed in the foamed polysaccharide by polyvalent metal cations and the foam subsequently treated with a strong acid in order to release carbon dioxide and to crosslink, by the cations that form, the polysaccharide while a dimensionally stable foam structure is formed. According to the printed publication, foams of a thickness of up to 5 mm can be stabilized in this manner. However, these thicknesses are insufficient in particular where subsequent cutting of the shaped articles made of foam into thinner layers is intended. Moreover, the formation of gases during manufacture leads to diffuculties in controlling the size of the pores and to great inhomogeneities in the foam.

Another process for the manufacture of alginate sponges is known from U.S. Pat. No. 3,653,383. Here, calcium alginate is at first produced from alginic acid and calcium carbonate, the calcium alginate formed is then ground, and the resulting gel is subjected to freeze-drying. Relatively large-format sponge-like materials can be produced in this manner, however, the product obtained disintegrate relatively quickly in water. Thus, the alginate sponges—in particular when cut into thin layers—have a wet-strength, in particular with regard to wet breaking strength, which is insufficient for cosmetic or medical pads.

The object of the present invention is therefore to provide relatively large-format, highly homogeneous shaped articles based on compounds of alginates and polyvalent metal ions, which have a high degree of wet-strength, in particular with regard to wet breaking strength, and which can be cut into thin layers using commonly-used cutting devices, and which have an attractive appearance, i.e. in particular a high degree of whiteness, and which can therefore be used in cosmetic or medical applications such as cosmetic skin pads or medical plasters etc. Furthermore, it makes possible the provision of homogeneous thick porous alginate layers, from which suitable cosmetic or medical forms of application that can also be administered orally can easily be manufactured by compressing and/or punching out, such as, for example, shaped articles for implants, satiation comprimates, means for the controlled, (i.e., retarded) release of active substances or the like.

The inventors of the present patent application succeeded, surprisingly, in providing homogeneous, relatively thick, large-format shaped articles based on alginates of polyvalent metal salts that can be obtained by special processes that are also the subject matter of the present invention, and which solve the above-mentioned problems of the shaped articles of the state of the art, and which are thus eminently suitable for the manufacture of cosmetic or medical products.

### DETAILED DESCRIPTION OF THE INVENTION

Thus, the present patent application provides a process for the manufacture of alginate-containing porous shaped articles comprising the steps:

a) Preparing an aqueous solution of a water-soluble alginate,

b1) Adding one or more salts of a polyvalent metal ion with a multidentate complexing anion to the aqueous solution of the water-soluble alginate, and shifting of the complex-formation equilibrium of the polyvalent metal ion and the multidentate complexing anion while increasing the available concentration of the polyvalent metal ion in the alginate solution, and thus formation of salts of the alginate with the above-mentioned polyvalent metal ion, or

b2) adding a multidentate complexing agent for a polyvalent metal ion to the aqueous solution of the water-soluble alginate and admixing one or more poorly water-soluble salts of a polyvalent metal ion

c) pouring the (still) flowable aqueous alginate composition in to a mould, and,

d) drying the aqueous alginate composition while a porous alginate-containing shaped article is formed.

Step (a). The water-soluble alginates used in step (a) preferably are alkali metal alginates such as alginates of sodium, potassium, etc. The underlying algin acid is a natural acid polysaccharide primarily extracted from so-called brown algae (Phaecophyceae) with a high molecular weight between 30,000 and 200,000, which contains chains formed from D-mannuronic acid and L-guluronic acid. The degree of polymerization changes depending on the kind of alga used for extraction, on the season during which the algae were collected, the geographic origin of the algae as well as the age of the plants. The main kinds of brown algae from which algin acid is obtained, are, for example Macrocystis pyrifera, Laminaria cloustoni, Laminaria hyperborea, Laminaria flexicaulis, Laminaria digitata, Ascophyllumnodosum and Fucus serratus. However, algin acid or alkali alginates can also be obtained microbiologically, for example by fermentation with Pseudomonas aeruginosa or mutants of Pseudomonas putida, Pseudomonas fluorescens or Pseudomonas mendocina, see. e.g. EP-A-251905 and Römpp Chemie Lexikon “Naturstoffe” (Encyclopedia of Natural Products) published by Thieme Verlag, 1997 and documents cited therein.

According to the invention, alginates with an average particle size of up to about 0.2 mm and a viscosity in aqueous solution of from 300 to 800 mPas (1% solution, pH 7, 20° C.) are preferred. According to the invention, sodium alginate is particularly preferred. The aqueous solution of the water-soluble alginate used in step (a) preferably has such a concentration, that, in the aqueous suspension formed according to step (b) a concentration is formed of 0.2 to 3 wt %, more preferably 0.3 wt % to 2.5 wt %, and still more preferably 0.4 wt % to 1.2 wt % of alginate in relation to the amount of water used. The solution can be prepared by suspending the desired amount of alginate in, e.g., distilled water. The concentration of the alginate in the aqueous suspension influences the hardness of the porous shaped articles formed. Concentrations of more than 2 wt % lead to relatively hard and brittle, respectively, shaped articles, which less preferred. Concentrations below 2 wt % lead to less brittle shaped articles, which is more preferred.

Step (b1). In step (b1), one or more salts of a polyvalent metal ion with a multidentate complexing anion are added to the aqueous solution of the water-soluble alginate obtained in step (a).

Such polyvalent metal ions are suitable which form poorly soluble compounds with the alginate used, i.e. which act as cross-linking metal ions. Such polyvalent metal ions include, for example, alkaline-earth metal ions and transition metal ions which form poorly soluble compounds with alginates. Alkaline-earth metal ions, such as beryllium, magnesium or calcium are preferred. Calcium is particularly preferred. Beryllium and magnesium are less preferred, since the former is not acceptable from a cosmetic point of view and since the cross-linking effect of the magnesium is small. Thus, calcium salts are particularly preferred according to the invention for they are physiologically and, particularly, cosmetically acceptable and have a strong cross-linking and/or gelation effect compared to alginates. In addition, e.g. barium, strontium, zinc, manganese, iron, aluminum can also be used.

According to the invention, the multidentate complexing anion in-the complex salt of the polyvalent metal ion is preferably a carboxylate of a polycarboxylic acid. Carboxylates of aliphatic dicarboxylic to tetracarboxylic acids, such as, for example citric acid (2-hydroxy-1,2,3-propanetricarboxylic acid), malic acid, oxalic acid, 1,3-propanetricarboxylic acid, agaric acid, ethylenediamine tetraacetic acid (EDTA), 1,2,3-propanetricarboxylic acid etc. are preferred.

Polycarboxylic acids that are physiologically tolerable, particularly tolerable for the skin, are particularly preferred. In particular, this includes carboxylates of α-hydroxypolycarboxylic acids such as citric acid.

Citrate, malate and the anion of the EDTA are particularly preferred as multidentate complexing anions. Citrates are most preferred.

According to the invention, calcium citrate (stoichiometry: Ca₃Citrate₂) is particularly preferred as the complex salt of a polyvalent metal ion with a multidentate complexing anion that is added in step (b1).

Addition of the complex salt of a polyvalent metal ion with a multidentate complexing anion in step (b1) may take place by admixing in solid or dissolved form.

Addition of the complex salt to the alginate solution expediently takes place in a temperature range of between 5° C. and 80° C., preferably, however, at room temperature (20° C.).

The amount of the complex salt added in step 1b) is expediently selected so, that the concentration of the complex salt in the resulting solution amounts to around 0.1 to 500 mmol/litre.

The amount of the added complex salt in relation to the amount of the alginate in the solution is preferably selected so that the molar ratio of the complex salt and the alginate amounts to about 0.001 to 0.1.

Subsequent to the addition of the complex salt, the shifting of the complex-formation equilibrium of the polyvalent metal ion and the multidentate complexing anion in the aqueous solution of the alginate takes place:

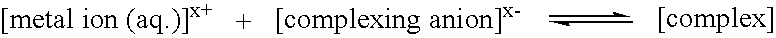

Of course, unequal charges of metal ion and complexing anion are possible, given appropriate stoichiometry, as in the calcium citrate system.

This equilibrium is commonly described by the so-called complex formation constant:

$${K}_{a} = \frac{[ Complex ]}{[ {Me}^{+} ] \cdot [ {A}^{-} ]} ,$$

where [Me⁺], [A⁻] and [Complex] are the concentrations (activities) of the polyvalent metal ion, the complexing multidentate anion and the complex in the solution, respectively. The complex formation constant is the inverse of the dissociation constant.

The complex formation constant yields information about the stability of the complex in the respective chemical environment, and therefore is also called stability constant of the complex. The larger the value of the constant is, the more stable the complex.

The shifting of the above-mentioned equilibrium in step (b1) carried out according to the invention takes place, for example, by reducing the concentration of the complexing anion in the solution. According to the equilibrium constant, the concentration of the uncomplexed polyvalent metal ion in the solution is thus increased. The shift of the equilibrium can be effected by a change, particularly by an increase in temperature, since the equilibrium constant depends on temperature, among other things. Also, an addition of another metal salt is conceivable which would have an influence on the balanced reaction complexed anion/free anion without, however, forming insoluble alginates.

However, shifting of the equilibrium is preferably effected by a reduction of the concentration of the free complexing anion in the solution, particularly preferably by the addition of at least one acid:

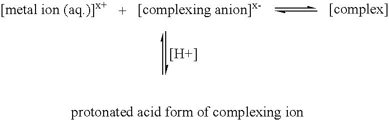

Preferably, the added acid is a stronger acid than the conjugated acid of the complexing anion, and is therefore capable of protonating it. However, the conjugated acid itself can also be added, such as citric acid in the case of citrate as an anion. Since the citrate, which results from the dissociation of the salt of the polyvalent cation such as, e.g. Ca²⁺, is formed in the form of Citrate³⁻, it is protonated in an aqueous solution by adding citric acid while hydrogen citrates are formed, and is thus withdrawn from the complex formation equilibrium. For example:

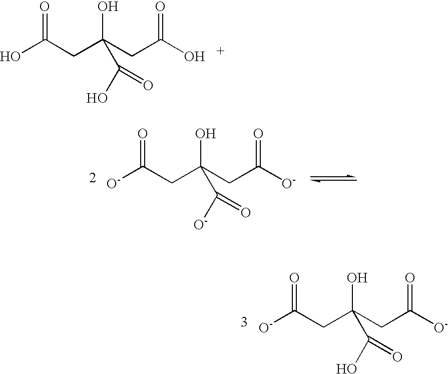

Preferred acids are, for example, inorganic mineral acids, such as hydrochloric acid, sulfuric acid, phosphoric acid or aliphatic carboxylic acids, such as acetic acid, etc.

The amount of the acid added depends on the complex salt used and its complex formation constant in aqueous solution. For example, it may amount to around 0.1 to 20 times (mol/mol) the concentration of the complex salt. In particular, the molar ratio of calcium citrate to an acid such as citric acid, preferably amounts to between 0.1 to 20, more preferably from 0.5 to 10.

As a rule, the a pH adjustment to less than about 6.0 is sufficient to shift the complex formation constant far enough for the concentration of the polyvalent metal salt to increase enough that the solubility product of the alginate salt is exceeded, i.e. that the insoluble alginate of the polyvalent metal salt precipitates or that the solution gels.

Surprisingly, it has been found, that the pH value adjusted in this step influences the breaking strength of the porous shaped articles obtained. In order to obtain a higher breaking strength, pH values of less than 6 are preferable, more preferably less than 5. These low pH values are particularly preferable in combination with a low alginate concentration of less than 2 wt % adjusted in step (b) in the total suspension.

The rate of formation of the insoluble alginate, and thus the flowability or pourability of the alginate solution or suspension, can be controlled very exactly and easily by the amount and rate at which the acid is added, as well as by temperature control if necessary, particularly because of the high diffusion rate of the protons in the aqueous solution. It is thus possible to obtain homogeneous shaped articles with high thicknesses of at least around 1 cm after drying, which have a sufficient wet-strength, in particular with regard to wet breaking strength, so that they can be used as cosmetic or medical sponge-like wet-strength materials, as described below, if necessary after subsequent cutting into thinner layers or by compressing and/or punching out.

Step (b2). In a further embodiment (step (b2)) of the process according to the invention, the further retardation—in comparison to the state of the art—of the formation of the insoluble alginate in the alginate solution which facilitates a more homogeneous incorporation of the polyvalent metal salt into the solution of the alginate and thus a homogeneous quality of the porous shaped article, takes place, not (as in the state of the art) by adding to the alginate solution of step a) a soluble salt of a polyvalent metal ion which forms poorly soluble salts with the alginates (such as calcium chloride), but rather by adding poorly soluble salts of these polyvalent metal salts, such as CaSO₄.

At first, a multidentate complexing agent for a polyvalent metal ion is added to the aqueous solution of the water-soluble alginate in step (b2). Naturally, the multidentate complexing agent is added in the form of an ionic compound or as a covalent compound, e.g. in the form of a conjugated acid. The multidentate complexing agent can be added to the solution of the alginate in solid or dissolved form. In principle, the above-mentioned complexing agent can be salts of polyvalent metal ions that form poorly soluble alginates, as well as salts of monovalent or polyvalent metal ions that do not form poorly soluble compounds with alginates. Mixtures of such metal salts can also be used. Salts of monovalent or polyvalent metal ions that do not form poorly soluble compounds with alginates (such as sodium citrate, or its conjugated acids, such as, e.g. citric acid) are preferred, since the retarding effect of the multidentate anion on the formation of free polyvalent metal ions, which can serve to form poorly soluble alginates, is more pronounced. In principle, however, the salts of polyvalent metal ions with multidentate complexing anions used in step (b1), such as e.g. calcium citrate, can also be added.

In this and other variants, acid is added if necessary, after or during mixing a poorly soluble metal salt of a polyvalent metal ion such as e.g. calcium sulfate, in order to increase the concentration of free metal ions that form poorly soluble compounds with alginates and to accelerate the homogeneous cross-linking of the alginates. Preferred acids are, for example, inorganic mineral acids such as, e.g., hydrochloric acid, sulfuric acid, phosphoric acid or aliphatic carboxylic acid, such as, e.g., acetic acid. Particularly preferred is hydrochloric acid.

Also in the variant of step (b2), it can be seen that the set pH value has an influence on the breaking strength of the porous shaped articles obtained. Therefore, in order to obtain a higher breaking strength, a pH value of less than 6 is preferred, more preferably of less than 5, also in step (b2). Again, these low pH values are particularly preferable in combination with a small alginate concentration of less than 2 wt % adjusted in step (b) in the total suspension. The adjustment of the pH value, in principle, may also take place by a prior addition of an acid, such as HCl to the alginate solution or the alginate solution to which a complexing agent has been added, such as sodium citrate or citric acid, and subsequent addition of the poorly soluble metal salt, such as CaSO₄.

The concentration of the added multidentate complexing agent for a polyvalent metal ion amounts to about 0.0001 to 1 mol/liter, preferably from 0.001 to 0.5 mol/liter. The molar ratio of the amount of the water-soluble alginate in relation to the molar amount of the added multidentate complexing agent for a polyvalent metal ion amounts to preferably 0.0001 to 1, more preferably 0.001 to 0.5.

The polyvalent metal ions that are added in the form of their poorly soluble salts in the second adding step of step (b2) are those metal ions that form poorly soluble salts with alginates or cross-linked alginates, and in this regard, we may refer to the salts mentioned in step (b1). In principle, the corresponding anions can be selected arbitrarily, however, in water, they must form poorly soluble salts with the polyvalent metal ions or cations. Here, calcium salts are also preferred, particularly calcium sulfate. CaCO₃ and other carbonates are less preferred, since CO₂ may form in the preferably acid conditions of the preparation of the poorly soluble alginate, which makes controlling the reaction or the qualities of the alginate-containing shaped body more difficult.

The solubility, in water at 20° C., of the poorly water-soluble salt of the polyvalent metal ion added in step (b2) preferably amounts less than 10 g/liter, more preferably 5 g/liter, still more preferably 0.1 to 3 g/liter. If the solubility is higher, a more rapid formation of the poorly soluble alginates may occur which leads to a reduction of the possible processing time and thus, to an inhomogeneous product. If solubility is less than the above-mentioned range, the formation of the poorly soluble or cross-linked alginates may take place too slowly, which is also undesirable.

By admixing further salts, in particular such salts that do not form poorly soluble alginates, such as e.g. sodium sulfate, sodium chloride etc., the solubility of the poorly water-soluble salts of polyvalent metal ions can be reduced even further, and thus, the processability or homogeneity can be enhanced.

The amount of the poorly soluble salt of the polyvalent metal ion is expediently selected, so that the concentration of the salt in the resulting solution is between about 0.1 and 500 mmol/litre, whereby, in this case, the total amount of the salt in relation to the volume of the solution is meant, even if the salt does not dissolve completely.

The amount of the added poorly soluble salt of the polyvalent metal ion in relation to the amount of the soluble alginate is preferably selected so that the molar ratio of the alginate to the poorly soluble of the polyvalent metal ion is between 0.001 and 1.

The amount of the added poorly soluble salt of the polyvalent metal ion in relation to the amount of the submitted multidentate complexing agent is preferably selected so that the molar ratio of the poorly soluble salt of the polyvalent metal ion and the multidentate complexing agent is between 0.1 and 10.

According to the two process variants (b1) and (b2), the formation of the poorly soluble alginates is expediently controlled so that the increase of the concentration of the uncomplexed divalent metal ion is so small, that a flowability of the alginate solution, expressed as viscosity at room temperature (20° C.), of under about 1000 mPas is made possible for at least 1 minute, preferably for about 2 minutes, still more preferably for about 3 minutes.

The formation of the alginate gels or the mixing, respectively, according to steps (b1) and (b2) is preferably carried out in mixers with stator/rotor system, such as, e.g., a colloid mill.

Step (c). Pouring the (still) flowable alginate composition into a mold desired for later drying can take place in a known manner. Herein, layer thicknesses of the flowable alginate composition of up to 50 cm are possible. Preferred shapes are box shapes with a rectangular layout. Pouring can take place at any suitable stage of the process. For example, the solution of the water-soluble alginate from step (a) may already be poured into the mould used for later drying if a sufficiently thorough mixing can be ensured in this mould. Preferably, however, pouring takes place after cross-linking or the precipitation of the poorly soluble alginate in step (b1) or (b2) has been initiated.

Step (d). Drying in step (d) takes place in a known manner. Freeze-drying is particularly preferred. This can also take place in a known manner, and, for example, DE 4328329 C2 or DE 4028622 C2 can be referred to in this context, which shall expressly be referred to with regard to step (d) of the process according to the invention, and which are thus part of the process according to the invention.

In a preferred embodiment of the process according to the invention, addition of at least one other component takes place prior to step d), in particular prior to step c), the component being selected from a group consisting of: cosmetic or medical active substances, further natural or synthetic hydrocolloid-forming polymers and cosmetic or medical adjuvants or additives.

Further natural or synthetic hydrocolloid-forming polymers include (partially) water-soluble, natural or synthetic polymers that form gels or viscous solutions in aqueous systems. They are expediently selected from further natural polysaccharides, synthetically modified derivatives thereof or synthetic polymers. Further polysaccharides include e.g. homoglycans or heteroglycans such as, for example, carrageenan, pectins, tragacanth, guar gum, carob-bean gum, agar, gum arabic, xanthan gum, natural and modified starches, dextrans, dextrin, maltodextrins, chitosan, glucans, such as β-1,3-glucan, β-1,4-glucan, such as cellulose, mucopolysaccharides, such as, in particular hyaluronic acid etc. Synthetic polymere include e.g.: cellulose ethers, polyvinyl alcohol, polyvinyl pyrrolidone, synthetic cellulose derivatives, such as methylcellulose, carboxycellulose, carboxymethylcellulose, in particular sodium carboxymethycellulose, cellulose esters, celluloses ethers such as hydroxypropylcellulose, polyacrylic acid, polymethacrylic acid, poly(methyl methacrylate) (PMMA), polymethacrylate (PMA), polyethylene glycols etc. Mixtures of these polymers may also be used. However, those of hydrocolloid-forming proteins, such as e.g. collagen, are not preferred, since some consumers increasingly prefer the use of products of purely vegetable origin, in particular in cosmetics.

According to the invention, hyaluronic acid and/or its salts and/or their derivatives are particularly preferably added. Hyaluronnic acid is a highly viscous glucosaminoglycan with alternating β₁₋₃ glucuronic acid and β₁₋₄-glucosamine moieties; its molecular weight lies between 50000 and several million. Hyaluronic acid is often used as a sodium salt, e.g. in therapy, mainly in ophthalmology, surgery and in cosmetics. The salts of the hyaluronic acid, which are formed with alkaline ions, alkaline-earth ions, magnesium ions, aluminum ions, ammonium ions or substituded ammonium ions, can be used as carriers for increasing absorption of medicaments, see. e.g. Römpp Chemie Lexikon “Naturstoffe” (Encyclopedia of Natural Products) published by Thieme Verlag, 1997 and documents cited therein. According to the invention, sodium hyaluronate with a molecular weight of about 1,000,000 to 2,500,000 is particularly preferred. Addition of the hyaluronic acid to the process according to the invention leads, totally surprisingly, to a increased whiteness of the obtained alginate-containing porous shaped articles, in particular in the process variant (b1), but also in process variant (b2). For aesthetic reasons, this is particularly very much preferred in cosmetic applications. Moreover, hyaluronic acid also develops its therapeutic effect in particular in topical or local application, such as e.g. moisturization of the skin or support of wound healing.

The hyaluronic acid or its salts are added to the alginate-containing porous shaped articles according to the invention in an amount of about 0.1 to 90 percent by weight, preferably up to about 70 wt %, relative to the dried shaped article.

In a further preferred embodiment, the porous shaped articles according to the invention comprise carboxymethylcellulose, in particular sodium carboxymethylcellulose. The addition of sodium carboxymethylcellulose, surprisingly, leads to an improvement of the optical density of the porous shaped articles according to the invention without increasing the hardness or brittleness of the shaped articles. On the contrary, the addition of sodium carboxymethylcellulose leads to an improvement of the flexibility of the porous shaped articles obtained. Furthermore, the addition carboxymethylcellulose, in particular sodium carboxymethylcellulose, leads to a stabilization of the shaped articles. During the manufacture of the carboxymethylcellulose-containg shaped articles, the carboxymethylcellulose, in particular sodium carboxymethylcellulose, surprisingly prevents the sedimentation of the poorly soluble salt, in particular of the CaSO₄. The carboxymethylcellulose, in particular sodium carboxymethylcellulose, can be present in the shaped articles according to the invention in an amount of up to 90 wt % relative to the dry content of the shaped article. This corresponds to preferred ranges that are to be set in the aqueous suspension of up to 3 wt %, preferably 0.2 wt % to 3 wt %.

A preferred embodiment of the shaped articles according to the invention comprises the carboxymethylcellulose, in particular sodium carboxymethylcellulose, and hyaluronic acid and/or their salts and/or their derivatives.

In particular, active substances added include cosmetic or therapeutic or pharmaceutical active substances, particularly active substances suitable for external application. Preferably, the shaped article manufactured according to the invention contains at least cosmetic and/or pharmaceutical active substance. Accordingly, the shaped articles preferred according to the invention preferably are cosmetic or therapeutic active substances. Cosmetic shaped articles or shaped articles manufactured using cosmetic active substances within the sense of the invention are essentially active substances within the sense of the Lebensmittel-und Bedarfsgegenständegesetzes (LMBG) (German Foostuffs and Commodities Act), i.e., substances or preparations derived from substances that are intended to be applied externally on humans for the purpose of cleansing, grooming, or for the purpose of influencing appearance or body odor, or for the purpose of conveying impressions of odors, unless they are predominantly intended for relieving or eliminating diseases, ailments, physical defects or pathological complaints. In this sense, the cosmetic shaped articles manufactured in accordance with the invention are, for example cosmetic applications such as, e.g., face masks etc., which can serve as skin-washing and skin-cleansing agents, skin-care products, in particular skin-care products for the face, eye cosmetics, lip-care products, nail-care products, foot-care products, as well as hair-care or dental-care products. Examples of cosmetically effective compounds, or optionally e.g. dermatologically, therapeutically effective compounds, include: anti-acne agents, antimicrobial agents, antiperspirants, astringent agents, deodorizing agents, depilatories, conditioning agents for the skin, skin-smoothing agents, agents for increasing the hydration of the skin, such as e.g. glycerin or urea, sun-screening agents, keratolytics, radical-interceptors for free radicals, antiseptic substances, agents for the treatment of the symptoms of ageing of the skin and/or agents that modulate the differentiation and/or proliferation and/or pigmentation of the skin, vitamins such as vitamin C, agents with irritating side-effects, such as e.g. alpha-hydroxy acids, β-hydroxy acids, alpha-keto acids, β-keto acids, retinoids (retinol, retinal, retinic acid), anthralines (dioxyanthranol), anthranoids, peroxides (in particular, benzoyl peroxide), minoxidil, lithium salts, antimetabolites, vitamin D and its derivatives; catechols, flavonoids, ceramides, fatty substances, such as mineral oilks, such as paraffin oils or Vaseline oils, silicone oils, vegetable oils such as coconut oils, sweet almond oil, apricot oil, corn oil, jojoba oil, olive oil, avocado oil, sesame oil, palm oil, eucalyptus oil, rosemary oil, lavender oil, pine oil, thyme oil, mint oil, cardamom oil, orange-blossom oil, soybean oil, bran oil, rice oil, rapeseed oil and castor oil, wheat-germ oil and vitamin E isolated thereform, evening-primrose oil, vegetable lecithins (e.g. soybean lecithin), sphingolipids/ceramides isolated from plants, animal oils or fats, such as tallow, lanolin, butyric oil, fatty-acid esters, esters of fatty alcohols, and waxes with a melting point corresponding to skin temperature (animal waxes such as beeswax, carnauba wax and candelilla wax, mineral waxes, such as microcristalline waxes, and synthetic waxes, such as polyethylene waxes or silicone waxes), as well as all oils that are suitable for cosmetic purposes, such as, for example, those mentioned in the CFTA treatise entitled Cosmetic Ingredient Handbook, 1^{st} edition, 1988, The Cosmetic, Toiletry and Fragrance Association, Inc., Washington, polyunsaturated fatty acids, essentially fatty acids (e.g. γ-linolenic acid), enzymes. coenzymes, enzyme inhibitors, hydrating agents, skin-soothing agents, detergents or foam-producing agents, and inorganic or synthetic matting fillers, abrasive agents. Moreover, plant active-substance extracts or essences obtained therefrom or individual substances may be mentioned, that can be added to the porous shaped bodies manufactured according to the invention. Generally, the plant active-substance extract is selected, as a rule, from the group consisting of solid plant extracts, liquid plant extracts, hydrophilic plant extracts, lipophilic plant extracts, individual plant constituents, and also mixtures thereof, such as flavonoids and their aglycones: rutin, quercetin, diosmin, hyperoside, (neo)hesperidin, hesperitin, Ginkgo biloba (e.g. ginkgo flavone glycosides), Crataegus extract (e.g. oligomeric procyanidines), buckwheat (e.g. rutin), Sophora japonica (e.g. rutin), birch leaves (e.g. quercetin glycosides, hyperoside and rutin), elderflowers (e.g. rutin), lime blossoms (e.g. ethereal oil with quercetin and farnesol), hypericum oil (e.g. olive-oil essence), calendula, arnica (e.g. oleaginous essences of the flowers with ethereal oil, polar essences with flavonoids), melissa (e.g. flavones, ethereal oil); immunostimulants: Echinacea purpurea (e.g. alcoholic essences, fresh plant juice, pressed juice), Eleutherokokkus senticosus; alkaloids: rauwolfia (e.g. prajmaline), myrtle (e.g. vincamine); other phytopharmacons: aloe, horse chestnut (e.g. aescin), garlic (e.g. garlic oil), pineapple (e.g. bromelain), ginseng (e.g. ginsenosides), sow-thistle fruits (e.g. extract standardised with respect to silymarine), butcher's-broom root (e.g. ruscogenine), valerian (e.g. valepotriates, tct. valerianae), kava kava (e.g. kavalactones), hop flowers (e.g. hop bitters), extr. passiflorae, gentian (e.g. ethanolic extract), anthraquinone-containing tinctures, e.g. aloin-containing aloe-vera juice, pollen extract, algae extracts, liquorice-root extracts, palm extract, galphimia (e.g. mother tincture), mistletoe (e.g. aqueous ethanolic essence), phytosterols (e.g. β-sitosterol), mullen flowers (e.g. aqueous alcoholic extract), drosera (e.g. liqueur-wine extract, sea-buckthorn fruits (e.g. juice obtained therefrom or sea-buckthorn oil), marshmallow root, primrose-root extract, fresh plant extracts from mallow, comfrey, ivy, horsetail, yarrow, ribwort (e.g. pressed juice), stinging nettle, celandine, parsley; plant extracts from Norolaena lobata, Tagetes lucida, Teeoma siems, Momordica charantia and aloe-vera extracts.

Preferred cosmetic active substances are natural and synthetic moisturising factors such as, for example, glycerin, urea and ceramides, skin-protecting agents, skin lighteners, vitamins, antioxidants, so-called anti-ageing agents, anti-irritation agents, sun-screening agents, etc.

Further preferred cosmetic active substances are natural fats and oil, i.e. triglycerides of natural fatty acids, e.g. because of the moisturizing effect on the skin.

A particularly preferred cosmetic active substance is urea, which is thought to have the effect of a local anesthetic.

As distinct from the shaped articles described above, which are essentially used in the cosmetic field, in the case of the shaped articles that are used therapeutically (medicaments) it is a question of those which contain at least one pharmaceutical or therapeutic, in particular also dermatological, active substance and which in the sense of the Arzneimittelgesetz (German Drug Law) are intended, inter alia, to cure, relieve or prevent diseases, ailments, physical defects or pathological complaints. Such agents or active substances are intended for external application, in which case it may be a question of dermally active substances but also of transdermal active substances. They include, for example: agents for the treatment of skin diseases, externally applicable analgesics, e.g. dextropropoxyphene, pentazocine, pethidine, buprenorphine; antirheumatics/antiphlogistics (NSAR), e.g. indomethacin, diclofenac, naproxen, ketoprofen, ibuprofen, flurbiprofen, salicylic acid and salicylic-acid derivatives such as acetylsalicylic acid, oxicams; steroid hormones, e.g. betamethasone, dexamethosone, methylprednisolone, ethynyl estradiol, medroergotamine, dihydroergotoxine; gout remedies, e.g. benzbromarone, allopurinol; external dermatological agents, including antibacterial agents, antimycotics, antiviral active substances, antiinflammatory active substances, antipruritic active substances, anaesthetising active substances, e.g. benzocaine, corticoids, anti-acne agents, antiparasitic active substances; externally applicable hormones; venous therapeutic agents; immunosuppressives etc., all for external application.

Preferred therapeutic agents are analgesics, e.g. immunosuppressives, hormones, agents for the treatment of skin diseases such as neurodermatitis, atopic dermatitis etc., and anti-herpes agents.

Moreover, the porous shaped articles manufactured according to the invention may contain one or more auxiliary substances. Auxiliary substances include: fillers, pH-adjustment agents, such as buffering substances, stabilizers, co-solvents, pharmaceutically and cosmetically conventional or other dyestuffs and pigments, preservatives, plasticizers, lubricants and slip additives. etc. Squalane is a particularly preferred auxiliary substance. Squalane has a soothing and smoothing effect on the skin.

Moreover, the invention relates to the use of a salt of a polyvalent metal ion with a multidentate complexing anion for the manufacture of porous alginate-containing shaped articles. This means that such a salt is added as such during the formation of such shaped articles, and is not formed partially or completely at any stage of the manufacture of such shaped articles.

By means of the present invention, porous shaped articles containing alginate of polyvalent metal ions can be manufactured which have a thickness of at least one centimeter, preferably at least 2 cm, and which are obtained by cross-linking (or precipitating) of alginate-containing aqueous solutions with salts of polyvalent metal ions and subsequent drying of the aqueous suspension of the obtained cross-linked alginate. Herein, the thickness of the shaped article means the shortest distance between two points in such a shaped article. The manufacture of such thick large-format shaped articles with the desired wet-strength, particularly with the desired wet breaking strength, capability of being cut, etc., was not possible in the state of the art until now. These porous shaped articles are preferably obtained through the process according to the invention. The processes comprising the freeze-drying of ground insoluble alginates lead to easily disintegrating porous or sponge-like materials unsuitable for the presently intended use.

When suspending 1 g of the shaped article in 100 g of water at 20° C., the porous shaped articles according to the invention have a pH value of the aqueous phase of less than 7, preferably less than 6. Such an acid pH is preferred in particular in cosmetic application on the skin.

The porous shaped article according to the invention preferably has a density of 0.005 to 1 g/cm³, preferably from 0.01 to 0.5 g/cm³ (determined according to DIN 53420).

The porous shaped article according to the invention preferably has a wet-strength of at least about 10 mN/mm layer thickness (determined according to DIN 53328).

The porous shaped article according to the invention does not consist of and does not essentially consist of spun alginate fibers, such as e.g. of calcium alginate fibers.

The above-mentioned porous shaped articles according to the invention can, as has been mentioned above, additionally contain at least one further component selected from the group including: cosmetic or medical active substances, further natural or synthetic hydrocolloid-forming polymers and cosmetic or medical adjuvants or additives. These may be contained in the porous shaped articles according to the invention in amounts of up to 0.75 g/g, preferably less than 0.5 g/g of the porous shaped article.

The above-mentioned porous shaped articles according to the invention are eminently suitable for the manufacture of layered shaped articles by cutting the porous shaped articles according to the invention in the manner known. For example, this is not possible with the sponge-like materials obtained by freeze-drying of ground insoluble alginates. By cutting the porous shaped articles according to the invention, layer thicknesses of, for example, 0.5 to 20 mm are obtained. The invention also relates to the layered porous shaped articles thus obtained. Such layered porous shaped articles are particularly suitable for external application, such as cosmetic or medical pads, such as material for wound dressing, primary wound dressing, implant material, and cell cultivation matrix.

Furthermore, the porous shaped articles according to the invention are eminently suitable for the manufacture of compressed, expandable, sponge-like shaped articles as described, for example, in the applicant's EP 0901792, based on collagen. They can be easily manufactured from the large-format porous shaped articles, which were obtained in particular after freeze-drying, by punching out and/or compressing, in particular on an industrial scale, which, until now, is not possible without problems according to the processes of the state of the art.

Such comprimates are suitable in particular for oral, buccal or nasal application, such as, for example, satiation comprimates, which may additionally contain active substances, substances for dietary supplements or vitamins (e.g. DE 19942417).

In addition, because of the poorly soluble properties of the porous shaped articles according to the invention, they are suitable for the manufacture of forms loaded with active substances, in which the active substance is released in a controlled, in particular retarded, manner. Such forms include sponges containing active substances, such as implants, vaginal suppositories, as well as forms that can be administered orally, the latter especially as comprimates that expand to several times their compressed volume in a moist state, and that release the active substance contained from the sponge-like matrix (e.g.. WO 98/09617).

Furthermore, the present invention relates to porous shaped articles comprising alginates of polyvalent metal ions and hyaluronic acid and/or their salts and/or their derivatives. As explained above, these shaped articles, completely surprisingly, have an increased degree of whiteness which is very much preferred particularly in cosmetic, but also in medical application. With regard to the composition of such hyaluronic acid-containing porous shaped articles, we may refer to the above explanations. The hyaluronic acid-containing porous shaped articles are preferably manufactured according to the process according to the invention.

Furthermore, the present invention relates to the use of the porous shaped articles according to the invention or the shaped articles obtained by the process according to the invention as cosmetic agents. Preferably, the porous shaped articles contain, for cosmetic application, alginates of polyvalent metal ions and hydroxycarboxylic acids, in particular hydroxypolycarboxylic acids such as, in particular, citric acid, which can be added, in the shape of the above-mentioned multidentate complexing agent, already during the manufacture of the porous shaped articles according to the invention.

The use in cosmetics of the porous shaped articles according to the invention preferably takes place in the shape of cosmetic skin pads that are applied to the skin in moistened form and are taken off after a certain exposure time, for example after the active substances contained therein have been absorbed. The alginate itself also has a cosmetic effect such as hydration and smoothing of the skin.

Furthermore, the present invention relates to the use of the porous shaped articles according to the invention or the shaped articles obtained by the process according to the invention for the manufacture of a medical product. Such medical products include, for example, wound dressings, transdermal dressings, wound plasters, implants, substrates for cultivating cells, means for the controlled, in particular retarded, administering of active substances in the form of said implants, but also as a preparation to effect such retardation that can be administered orally, or as so-called satiation comprimates that have a satiation effect because of the expansion of the compressed porous shaped article in the stomach. The latter may also be loaded with dietary supplements, vitamins, minerals or other active substances.

The porous shaped articles according to the invention or the shaped articles obtained by the process according to the invention preferably serve the purpose of external application, such as, in particular, cosmetic or medical pads. Additionally, as has been mentioned, oral, buccal, vaginal, nasal application etc. is possible. As has been said, the homogeneous thick porous shaped articles made of alginates provided according to the invention permit the manufacture of any of those forms of application on an industrial scale with known processes, such as cutting, pressing, or compressing and/or punching out.

Particularly preferred shaped articles contain, relative to the dried substance, i.e. without residual moisture:

about 6 to 100 wt % alginate

0 to about 90 wt % carboxymethylcellulose, in particular the sodium salt thereof,

0 to about 70 wt % hyaluronic acid and/or the salts thereof and/or the derivatives thereof,

0 to about 90 wt % natural or synthetic oils,

0 to about 70 wt % citric acid or the salts thereof,

the latter correspond to the preferred ranges in the aqueous suspension in step (c), to be freeze-dried of: about 0.2 to 3 wt % alginate,

0 to about 3 wt % carboxymethylcellulose, in particular the sodium salt thereof,

0 to about 1 wt % hyaluronic acid and/or the salts thereof and/or the derivatives thereof,

0 to about 3 wt % natural or synthetic oils, and

0 to about 1 wt % citric acid or the salts thereof.

The shaped articles according to the invention, preferably have the form of a layer, i.e. length and width of the shaped articles are at least ten times, preferably 20 times as large as the thickness of the shaped article. Such layers can also be cut into certain shapes, e.g., in the form of a facial mask. The layers preferably have an area of at least 25 cm², more preferably at least 50 cm², still more preferred at least about 100 cm².

The invention also relates to laminates containing at least one layer described above, which is laminated on at least one side thereof with at least one further carrier layer. Preferably, the layer according to the invention is only laminated on one side, preferably with only one carrier layer. The carrier layer preferably consists of a Rayon web (viscose). Such laminates are preferably used as dressings or plaster for wounds, and are especially preferred as cosmetic masks.

The invention also relates to a combination containing at least one shaped article according to the invention as well as at least one aqueous solution that contains one or more active substances and/or adjuvants in matching spatial arrangement (application package, set, kit-of-parts, etc.). The solution of the active substances may be, e.g., solutions of readily volatile active substances or adjuvants that, because of the production process by freeze-drying, should not be or cannot be introduced into shaped articles, such as, e.g. certain portions of ethereal oils, perfumes, etc. Furthermore, the solution can also comprise pharmaceutical or cosmetic active substances. With regard to the following examples, the invention will be described in greater detail.

### EXAMPLES

### Example 1

(Manufacturing method 1: Calcium, complexed with multidentate ligands, thereafter shifting of equilibrium by addition of citric acid)

Step 1:

2500 g RO-water (desalinated water, reverse osmosis)

32.5 g sodium alginate

10.0 g calcium citrate

Work the alginate powder into the RO-water with a mixer until a homogeneous mixture is obtained. Then, stir in the calcium citrate (at this stage, cosmetic and/or medical active substances and/or oils or other substances expediently may be worked into the solution if needed.)

Step 2:

100 g RO-water

12.5 g citric acid

The citric acid is added to 100 ml RO-water under agitation.

Step 3: The solution of steps 1 and 2 are mixed intimately for about 30 seconds.

Step 4: The mixture from step 3 is poured into a mold and left to react for about 2 hours.

Step 5: The gelled shaped article is quick-frozen and freeze-dried.

Step 6: The freeze-dried, large-format, porous or sponge-like shaped article which, if necessary, is loaded with additional substances, can be prepared in the above-mentioned manner.

### Example 2

(Manufacturing Method 2: Addition of a poorly soluble calcium salt to a multidentate complexing agent.)

Step 1:

2500 g RO-water (desalinated water, reverse osmosis)

32.5 g sodium alginate

12.5 g citric acid

Work the alginate powder into the RO-water with a mixer until a homogeneous mixture is the result. Then, stir in the citric acid (at this stage, cosmetic and/or medical active substances and/or oils or other substances expediently may be worked into the solution if needed).

Step 2:

50 g RO-water

10.0 g calcium sulfate

The calcium sulfate is added to 50 ml RO-water under agitation.

Step 3: The solution of steps 1 and 2 are mixed intimately for about 30 seconds.

Step 4: The mixture from step 3 is poured into a mould and left to react for about 1 h.

Step 5: The gelled shaped article is quick-frozen and freeze-dried.

Step 6: The freeze-dried, large-format, porous or sponge-like shaped article which, if necessary, is loaded with additional substances, can be prepared in the above-mentioned manner.

1-51. (canceled)

52. A process for the manufacture of alginate-containing porous shaped articles comprising:
(a) one of
(1) contacting one or more salts of a polyvalent metal ion and a multidentate complexing anion with an aqueous solution of a water-soluble alginate, shifting the complex-formation equilibrium of the polyvalent metal ion and the multidentate complexing anion, increasing the available concentration of the polyvalent metal ion in the alginate solution, or
(2) addition of a multidentate complexing agent for a polyvalent metal ion to an aqueous solution of a water-soluble alginate, and adding one or more polyvalent metal salts which are poorly soluble in water,
(b) pouring the flowable aqueous alginate composition into a mold, and
(c) drying the aqueous alginate composition to form a porous shaped article.

53. The process of claim 52, wherein the water-soluble alginate is an alkali metal alginate.

54. The process of claim 52, wherein the polyvalent metal salt is at least one alkaline earth metal salt.

55. The process of claim 52, wherein the polyvalent metal salt is a calcium salt.

56. The process of claim 52, wherein the multidentate complexing anion or the complexing agent is a carboxylate of a polycarbonate acid.

57. The process of claim 52, wherein the multidentate complexing anion or the complexing agent respectively is selected from a citrate and a malate.

58. The process of claim 52, wherein the shifting of the complex-formation equilibrium of the polyvalent metal salt and the multidentate complexing anion is achieved by the addition of at least one acid.

59. The process of claim 52, wherein the poorly soluble polyvalent metal salt has solubility in water of less than 10 g/liter at 20° C.

60. The process of claim 52, wherein the poorly soluble polyvalent metal salt is calcium sulfate.

61. The process of claim 52, wherein the drying is freeze-drying.

62. The process of claim 52 further comprising, prior to (d), the addition of a further component selected from the group consisting of cosmetic substances, medical substances, hydrocolloid-forming polymers, cosmetic adjuvants and medical adjuvants.

63. The process of claim 62, wherein the further component is selected from the group consisting of a polysaccharide, hyaluronic acid or a salt thereof, carboxymethylcellulose or a salt thereof, urea, squalane, and combinations thereof.

64. A porous, shaped, article having a thickness of at least one centimeter, comprising alginate polyvalent metal ions, and the cross-linked alginate obtained by cross-linking alginate-containing aqueous solutions with polyvalent metal salts and subsequent drying of the aqueous suspension.

65. The article of claim 64, further comprising the addition of at least one further component selected from the group consisting of cosmetic substances, medical substances, hydrocolloid-forming polymers, cosmetic adjuvants and medical adjuvants.

66. The article of claim 64, further comprising a component selected from the group consisting of a polysaccharide, hyaluronic acid or a salt thereof, carboxymethylcellulose or a salt thereof, urea, squalane, and combinations thereof.

67. A process for the production of layered or compressed articles comprising cutting or compressing, respectively, the article of claim 64.

68. The process of claim 67, wherein the compressed or layered articles comprise orally administered preparations.

69. A cosmetic comprising porous shaped articles comprising alginates, polyvalent metal ions and hydroxycarboxylic acids.

70. A process comprising the topical, oral, buccal, vaginal, nasal, implant, or wound dressing application of a medical product including the article of claim 64.

71. A laminate comprising at least one layer comprising the article of claim 64, wherein at least one carrier layer is laminated to at least one side of the article.
